(19) United States
(12) Patent Application Publication
(10) Pub. No.: US 2003/0211179 A1
(43) Pub. Date: Nov. 13, 2003

(54) Method for improving psyllium functionality by solid-state reaction(s)

(76) Inventor: Liangli Yu, Fort Collins, CO (US)

Correspondence Address: Greenlee Winner and Sullivan, 5370 Manhattan Circle, Suite 201, Boulder, CO, 80303, US

(21) Appl. No.: 10/344,049

(22) PCT Filed: Aug. 8, 2001
(86) PCT No.: PCT/US01/24755

(51) Int. Cl. A23L 1/10; A61K 35/78; C12P 19/04

(52) U.S. Cl. 424/738; 435/101; 426/18

(57) ABSTRACT

This invention describes methods of producing psyllium with improved functionality by employing a solid-state enzyme reaction. The psyllium prepared according to the process disclosed herein show improved functionality, i.e., less gel hardness can thus be readily incorporated into a wide variety of products such as foodstuff, pharmaceutical compositions, animal feeds, beverages and the like.

### CROSS-REFERENCE TO RELATED APPLICATIONS

This application claims priority under 35 U.S.C. 119 (e) from U.S. Provisional Application No. 60/226,057, filed Aug. 17, 2000 which is incorporated in its entirety by reference herein.

### ACKNOWLEDGMENT OF FEDERAL RESEARCH SUPPORT

Not applicable.

### BACKGROUND OF THE INVENTION

This invention relates to methods of improving psyllium functionality by employing a solid-state enzyme reaction. The modified psyllium produced by this process exhibits less gelling and water uptake which makes it easier to disperse or mix with other ingredients than the staring raw psyllium in manufacturing products containing psyllium.

Psyllium is a mucilaginous material prepared from the seed husk from the plants of Plantago genus, which grows in certain sub-tropical regions. Psyllium is a highly branched acidic arabinoxylan [Fennedy et al., (1979) Carbohydrate Research 75:265-274]. The xylan backbone has both (1_{→}4) and (1_{→}3) linkages. Other monosaccharides present in psyllium include D-rhamnose, D-galactose, D-galacturonic acid, 4O-methyl-D-glucuronic acid, and 2-O-(2-D-galactopyranosyluronic acid)-L-rhamnose [Chan and Wypyszyk, (1988) Cereal Foods World 33:919-922].

A number of studies have shown diverse health benefits of psyllium [Chan and Wypyszyk, (1988) supra]. These include cholesterol-lowering activity, laxative effects, reduction of the risk of colon cancer, ease of gastric hypoacidity, and weight control [Park et al., (1997) Cereal Chemistry 74:207-211; Hara et al., (1996) Nutritional Biochemistry 7:549-554; Ganji and Kies, (1996) J. of Food Science and Nutrition 48:595-597; Arjmandi et al., (1992) J. of Nutrition 122:1559-1565; Anderson et al., (1990) Crit.Rev. in Food Science and Nutrition 29:95-146].

Recently, the cholesterol-lowering effect of psyllium has received more attention from food scientists because of its potential to add more value to the food. Coronary heart disease (CHD) is a major cause of morbidity and mortality for both men and women in the United States, with an estimated cost of $50-100 billion annually [Aygustin and Dwyer, (1999) Topics in Clin. Nutr. 10:1-13]. According to the National Cholesterol Education Program (NCEP) criteria, there are more than 60 million adult Americans still requiring cholesterol-lowering dietary treatment to reduce the total serum cholesterol and low-density lipoprotein (LDL) cholesterol [Jensen et al. (1993) J. of the Am. Col. of Nutr. 12:147-154; Aygustin and Dwyer, (1994) supra]. Dietary fibers, mainly water-soluble fiber, are generally considered to lower total and LDL cholesterol levels, and consequently reduce the risk of coronary heart diseases. Psyllium is an excellent source of both soluble and insoluble fibers. The soluble fiber content in psyllium is approximately eight times greater than that of oat bran. In 1998, the United States Food and Drug Administration (FDA) approved the labeling of food products containing a certain amount of psyllium to state they may have a claim of cholesterol-lowering effects. Thus, psyllium is an excellent food additive in functional foods to benefit human health. Similarly, the addition of psyllium to animal feed may result in better animal health and thus higher profit.

Functional foods have become an increasingly significant component of food research, development, and marketing. It has been widely accepted that eating is not as straightforward as it used to be. Diet can significantly alter the overall health and quality of life [Dreher, (1997) J. of Nutraceuticals, Functional & Medical Foods 1(2):3-5]. Food scientists and food manufacturers have paid more attention to the opportunities and challenges in the development of functional foods. Several factors may promote the market growth potential of functional foods in western countries. In this culture, food is generally separated from medicines or agents with health-improving properties. Aside from nourishment, factors that promote the concept of functional foods include: the increase in the aging population, interest in maintaining health, changing consumer habits (interest in self-treatment), successful use of functional foods to benefit health, increasing health care expenses, and changing governmental regulatory policies for foods. The successful marketing of functional foods promotes further interests of research and manufacture in this field. Some successful examples include “Knox-NutraJoint” [Nabisco Inc. Parsippany, N.J.], “Uncle Ben's Calcium Plus Rice” [Uncle Ben's Inc., Houston Tex.], “Benecol” (phytosterol ester products by McNeil's], and “Take Control” (phytosterol ester products by Lipton). The key for functional foods is developing bioactive additives. Psyllium became a very attractive candidate to be used as a nutraceutical in functional foods due to its cholesterol lowering effects, as well as its long-term safety.

In addition to its cholesterol-lowering effects, psyllium also has functional contributions in foods or other consumer products. For instance, psyllium may be used as a deflocculant in paper and textile manufacture, as an emulsifying agent, and as a binder or lubricant in meat products. Therefore, psyllium may have potential applications as a carbohydrate based fat-replacer to be used in low fat/low calorie foods.

However, the strong hydrophilic and gelling properties of psyllium make it difficult to incorporate psyllium in a food/beverage formula. The U.S. FDA requires that a considerable amount of psyllium must be included in a food product (about 10 g/day, equivalent of 7 g of soluble fiber/day) before a health claim can be made for reducing serum cholesterol. Psyllium can absorb as much as 90 times its weight in water. Because of this, a substantial amount of time is required for complete dispersal and miscibility of psyllium in an aqueous system containing other ingredients, including sugar, even with vigorous agitation [Rudin, (1988) U.S. Pat. No. Re.32,811]. An unpleasant slimy mouth feel is also related to these properties. Psyllium also has undesirable flavor characteristics that can be recognized in foods containing psyllium. Beverage is a preferred carrier of nutraceuticals. Adding the recommended amount of psyllium into a beverage formula is impossible due to its strong gel-forming capacity. To promote the application of psyllium in foods or other consumer products, it is necessary to improve its functional/biological/sensory properties.

There has been an extensive effort to improve the physicochemical/functional/sensory properties of psyllium. A method by Rudin (supra) generated a wide range of particle sizes to improve the dispersibility of psyllium powder. However, the psyllium prepared by this method did not become more dispersible than hose using substantially uniform particles. The sensory problems with psyllium could not be solved by this method.

Psyllium was mixed with other food ingredients such as sugar and malt, to improve the dispersibility [Wullschleger et al., (1993) U.S. Pat. No. 5,227,248]. The resultant psyllium preparations were not suitable for people who must restrict caloric intake. Also, this method has no significant effect on the total water consumption, gelling properties, and the sensory problems of the psyllium.

Powell et al. (1982) U.S. Pat. No. 4,321,263 disclosed a method of improving the dispersibility of psyllium preparations by coating and granulating psyllium with polyvinylpyrrolidone and polyethylene glycol. In 1988, Rudin (supra) reported that agglomeration of psyllium preparation in water could be prevented or reduced by coating psyllium with a food grade emulsifier.

Wullschleger et al. (supra) reported a novel psyllium preparation called psyllium nuggets. To make this preparation, psyllium was first blended with various combinations of wheat bran, corn bran, oat bran, different flours, sugar, high fructose corn syrup, gums, salts, and acids. The resultant blends were subjected to extrusion under certain conditions. The extruded nuggets were then used to make ready-to eat cereal products with an improved flavor and texture. The nuggets and ready-to-eat cereal products were reported to retain their cholesterol-lowering effects. However, the resultant psyllium preparation was not reported to have improved water absorbing and gelling properties.

Several U.S. patents [U.S. Pat. Nos. 4,915,960, 5,234,916, and 5,425,945] describe methods of blending certain acids with psyllium to reduce gelling properties. However, there is no evidence that the addition of acid could prevent the final gellation of psyllium containing products, or improve sensory properties.

Despite these efforts, the strong gelling and extreme water-uptake problems of psyllium persist and limit production of psyllium-containing products. In 1999, Yu, [WO99/623,42] and Yu et al. [WO99/63053] reported two new methods of modifying raw psyllium. However, these methods require that the modification reaction be carried out in either aqueous solution or in solvent, which necessitates an additional step of removing such solvent or buffer from the modified psyllium product prior to use. These methods may also generate hazardous wastes or require special equipment. Therefore, there is a present need for developing a simple method of modifying psyllium which can be readily adaptable for large scale production. To this end, the present invention provides a method of solid-state enzyme reaction to improve psyllium functionality. The solid-state reaction of the invention is simple and inexpensive, requiring only raw psyllium and an enzyme in sufficient amounts to produce a psyllium with desired properties. The modified psyllium thus prepared exhibits less gel hardness and adhesiveness and reduced water absorbing capacity than the staring material which makes the manufacturing of the psyllium containing products easier. The modified psyllium of the invention can be incorporated directly into a wide range of products without further treatment.

### SUMMARY OF THE INVENTION

The object of the present invention is to provide methods of producing psyllium with improved functionality by treating raw psyllium with an enzyme(s) in a solid-state reaction for a sufficient length of time such that the resultant modified psyllium has modified gelling properties and/or adhesiveness and/or water absorbing capacity than the starting raw psyllium.

Solid-state enzyme reaction of the invention is carried out with raw psyllium as a starting material and an enzyme preparation with no other liquid additives, i.e., no additional water or acid or solvent etc. In a preferred embodiment the enzyme was dissolved in a small volume of water and added to raw psyllium. The reaction can be carried out at a wide range of temperatures for a sufficient length of time, optimal for a given enzyme or enzymes to produce the desired modified psyllium. The reaction can be terminated by various means known in the art including heating.

The various parameters of the solid-state reaction such as the amount of enzyme(s), the type of enzyme, the reaction time, the reaction temperature, and the ratio of the enzyme to the starting raw psyllium and the like can be readily adjusted by a person of skill in the art to achieve maximal yield of psyllium product with desired functionality.

The enzyme(s) suitable for the solid-state reactions of the invention include but are not limited to xylanase, arabinase, cellulase, hemicellulase, pentosanase, beta-glucanase, and mixtures thereof. It is preferred that the enzymes are substantially free of amylase and protease activities. A majority of these enzymes are commercially available as mixtures as described in preferred embodiments.

In a preferred embodiment, raw psyllium was treated with a mixture of xylanase, arabinase, cellulase, hemicellulase, and beta-glucanase, at ambient temperature for approximately 120 hours. The resultant modified psyllium showed substantially modified gelling properties as measured by decreased gel hardness and adhesiveness, and decreased water absorbing capacity compared to the starting material.

In the it invention, the functionality of a psyllium preparation was measured by a degree of gel hardness and adhesiveness which are known to correlate with the properties of psyllium such as gelling capabilities, elasticity, and slimy mouth feel. Therefore, the modified psyllium of the invention has less slimy mouth feel and is easier to disperse and mix with other ingredients than is the raw psyllium.

The modified psyllium prepared by the method disclosed herein can be added directly after the solid-state reaction, for example, in preparing foodstuffs, livestock feeds, pharmaceuticals, bulk laxatives, drink mix, and beverages. Because the enzyme reaction is carried out in solid state, the only required additional processing of the product is inactivating the enzyme(s).

The solid-state enzyme reaction of the invention can also be used in the manufacture of a psyllium-containing product. In this case, a psyllium-containing food product can be prepared by blending raw psyllium with at least one edible ingredient, for example, eggs, flour, etc. to form a blend, and adding an effective amount of an enzyme under conditions to yield a final food product having an improved functionality compared to the starting raw psyllium. This eliminates the need to prepare modified psyllium prior to incorporation with other edible ingredients.

Also provided are methods of lowering serum LDL, triglyceride, and cholesterol in mammals by orally administering modified psyllium of the invention into a mammal.

### BRIEF DESCRIPTION OF THE DRAWINGS

FIG. 1 illustrates the improved hardness of the psyllium preparations treated according to the method described herein. The hardness was measured as described in the Examples Section. The X axis indicates a specific sample treated as follows: A−20,000 units Pentopan Mono BG; B−5,000 units Pentopan Mono BG+3,000 units shearzyme L; C−5,000 units Pentopan Mono BG+3,000 units shearzyme L in water; D−5,000 units Pentopan Mono BG+3,000 units shearzyme L and oven; E−5,000 units Pentopan Mono BG+3,000 units shearzyme L at 50C.; and F−control. The control is a sample with no enzyme added.

FIG. 2 illustrates the improved adhesiveness of the psyllium treated according to the method described herein. The adhesiveness was measured as described in the Examples Section. The X axis indicates a specific sample treated as follows: A−20,000 units Pentopan Mono BG; B−5,000 units Pentopan Mono BG+3,000 units shearzyme L; C−5,000 units Pentopan Mono BG+3,000 units shearzyme L in water; D−5,000 units Pentopan Mono BG 3,000 units shearzyme L and oven; E−5,000 units Pentopan Mono BG+3,000 units shearzyme L at 50C; and F−control. The control is a sample with no enzyme added.

FIG. 3 illustrates the improved hardness of the psyllium preparations treated according to the method described herein. The hardness was measured as described in the Examples Section. The X axis indicates a specific sample treated as follows: A−960 units viscozyme L; B−1,800 units viscozyme L; C−240 units viscozyme+1,800 units shearzyme L; and D−control. The control is a sample with no enzyme added.

FIG. 4 illustrates the improved adhesiveness of the psyllium treated according to the method described herein. The adhesiveness was measured as described in the Examples Section. The X axis indicates a specific sample treated as follows: A−960 units viscozyme L; B−1,800 units viscozyme L; C−240 units viscozyme+1,800 units shearzyme L; and D−control. The control is a sample with no enzyme added.

FIG. 5. illustrates the improved hardness of the psyllium preparations treated according to the method described herein. The hardness was measured as described in the Examples Section. The X axis indicates a specific sample treated as follows: A−1,100 units shearzyme L; B−5,000 units shearzyme L; C−control; and D−raw psyllium. The control is a sample with no enzyme added. The value shown in the raw psyllium column is the hardness measured in the raw psyllium without treatment.

FIG. 6 illustrates the improved adhesiveness of the psyllium treated according to the method described herein. The adhesiveness was measured as described in the Examples Section. The X axis indicates a specific sample treated as follows: A−1,100 units shearzyme L; B−5,000 units shearzyme L; C−control; and D−raw psyllium. The control is a sample with no enzyme added. The value shown in the raw psyllium column is the adhesiveness measured in the raw psyllium without treatment.

FIG. 7 shows improved gelling properties of modified psyllium by the solid-state reaction. The broken line represents raw psyllium, while the solid line represents the modified psyllium. Modified psyllium has reduced gelling capacity.

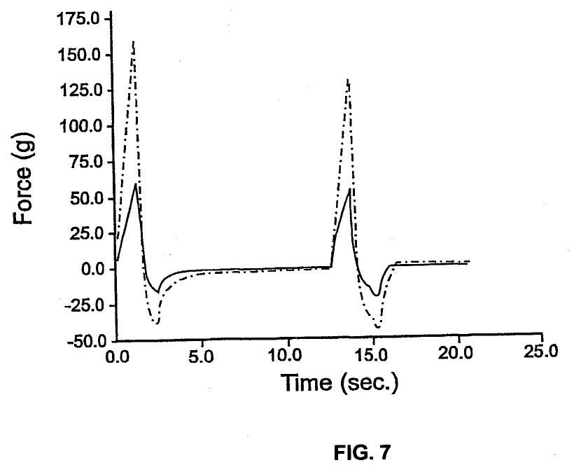

### DETAILED DESCRIPTION OF THE INVENTION

In general, the terms and phrases used herein have their art-recognized meaning, which can be found by reference to standard texts, journal references, and contexts known to those skilled in the art. The following definitions are provided to clarify their specific use in the context of the invention.

“Raw psyllium ” as used herein intended to include psyllium husk or any other product that contains psyllium husk or is derived from psyllium husk, e.g. whole psyllium seed, psyllium flour, isolated psyllium husk polysaccharides such as the soluble or insoluble fibers or mixtures thereof. Typical example of raw psyllium used in the invention is a commercial product of 98% purity and 40 mesh (JB Laboratories).

“Solid-state reaction” as used herein refers to a reaction condition which does not require additional liquid components such as water, buffer, acid, or solvent to modify raw psyllium to exhibit improved functionality. The solid-state reaction mixture contains raw psyllium and an effective amount of an enzyme(s) under conditions (temperature, the time period etc) to produce a desired modified psyllium. The “effective amount” of the enzyme is determined according to the desired functionality of the modified psyllium product, which in turn will depend on the intended end use. The effective amount of the enzyme will also vary depending on the length of the reaction time and the reaction temperature. The enzyme(s) is typically dissolved in a small volume (¹⁸ 15 ml) of water or buffer and added to the raw psyllium. The temperature can range from 4° C. to 55° C., preferably at ambient temperature (20-55° C.), and the length of reaction time can vary from hours to days sufficient to yield the modified psyllium with desired functionality. The reaction is terminated by various means known in the art including heating and treating with ethanol.

“Modified psyllium” as used herein refers to a psyllium preparation prepared by the solid-state enzyme reaction and exhibits a decreased gel hardness and/or adhesiveness compared to the starting psyllium. The modified psyllium preferably retains a substantial portion of the soluble and insoluble fiber content of the raw starting material.

The term “functionality” as used herein refers to the general physicochemical properties of the psyllium such as water absorption capacity and gelling property. These parameters are indicative of how easy it is to handle (i.e., mixing, dispersing etc) a given psyllium preparation in manufacturing other products. For example, in the present application, the gelling capacity of a psyllium preparation is measured as a degree of gel hardness and/or adhesiveness. It is well known in the art that these two parameters correlate with certain properties of psyllium such as gelling capacity, slimy mouth feel, elasticity and various sensory characteristics. Accordingly, “improved functionality” means that a psyllium preparation exhibits less gelling and/or water absorption after a process such as solid-state enzyme reaction. A psyllium product with “improved functionality” is thus expected to have more desirable properties, i.e., less slimy and easy to handle as an additive in a wide variety of products.

General procedure for Modification of Psyllium.

15 ml of individual enzyme preparation was mixed with 50 g raw psyllium (98% purity, 40 mesh, J B Laboratories). The mixture was kept at 20-50° C. for 120-150 hr in a 250 ml beaker covered with parafilm and aluminum foil. The reactions were terminated by heating at 85-90° C. for 25 min in a conventional oven or microwave for 1.5 min or ethanol treatment. The resulting psyllium preparations were further air-dried overnight if the reaction was terminated by ethanol treatment. The final product of solid-state reaction referred to herein as “modified psyllium” was obtained after passing the reaction product through a 20 mesh sieve.

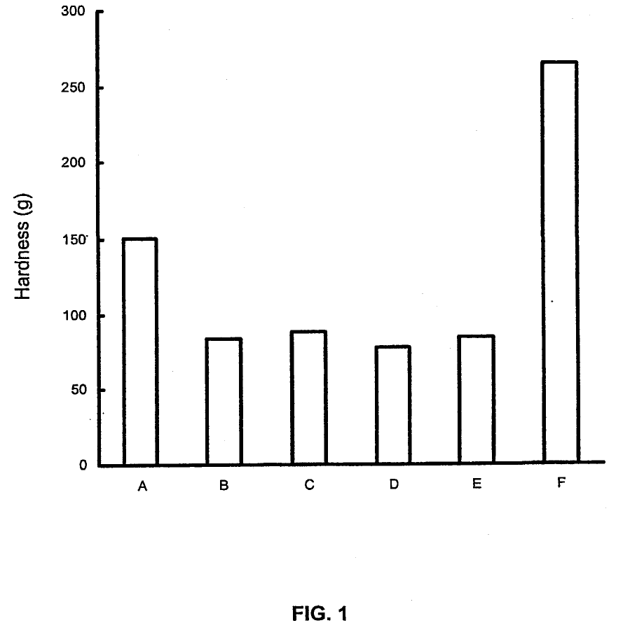

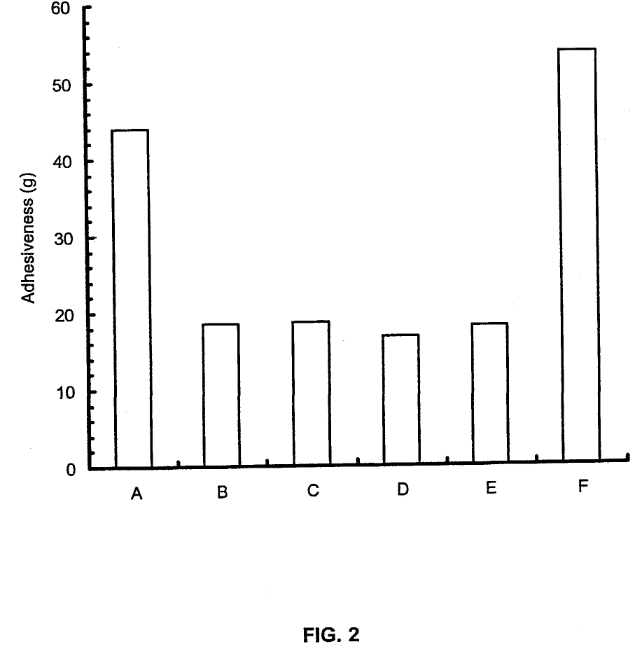

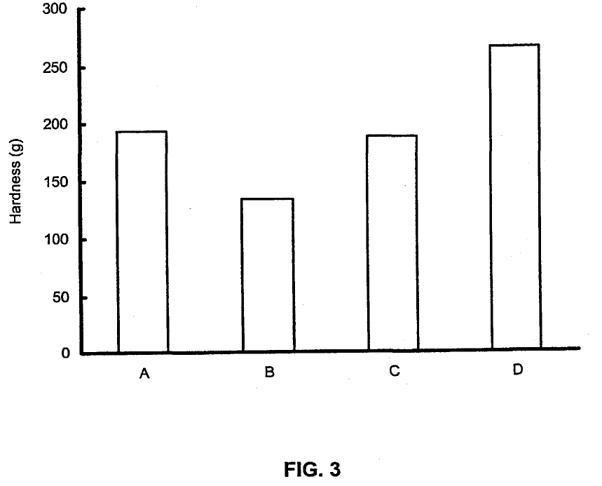

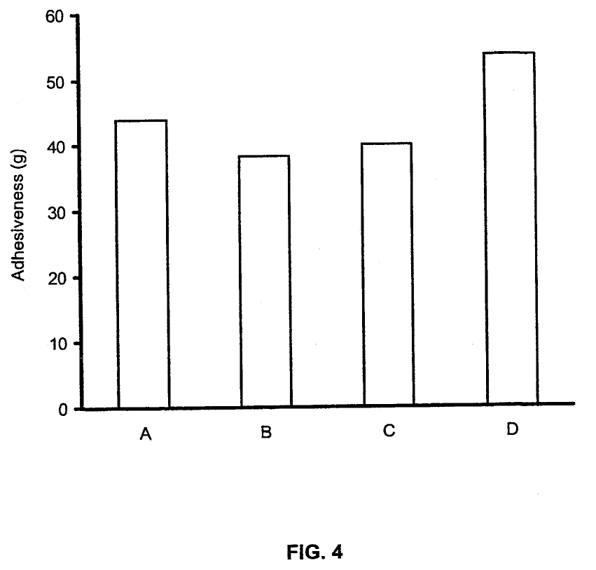

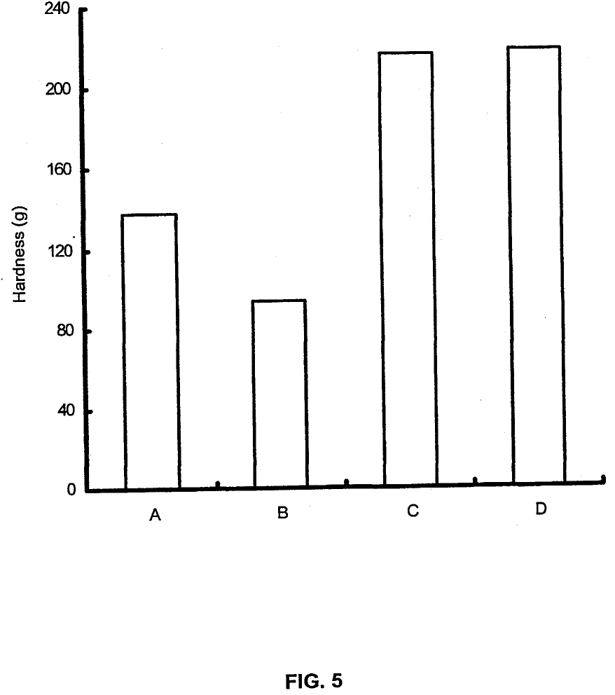

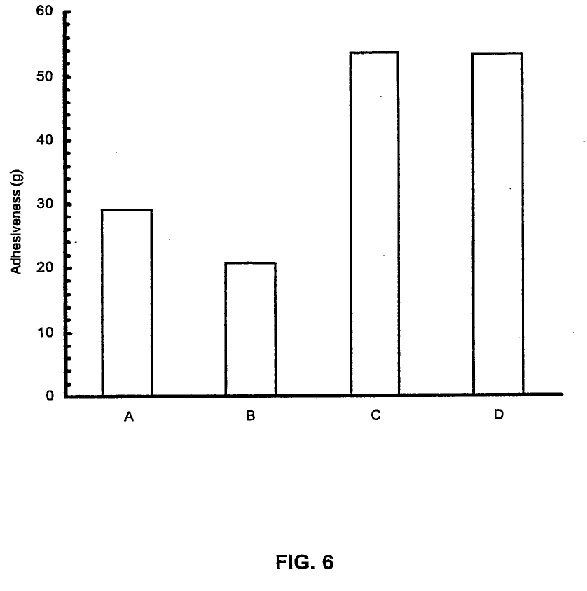

Controls of the solid-state enzyme reaction were performed using above procedure without addition of the enzyme(s). The analytical results of the control and raw psyllium are shown in Table 1, and used for comparison of all modified psyllium preparations obtained using the solid-state reactions. Control I was used for FIGS. 1-4 and control II was used in FIGS. 5 and 6.

Two tests, including water uptake rate and gelling properties, were performed to evaluate the functionality of the modified psyllium prepared by the solid-state enzyme reaction. The gelling properties measured and expressed as hardness and adhesiveness as described below are art-recognized predictors of functionality and sensory characteristics including slimy mouth feel of a psyllium preparation. Therefore, any psyllium preparation with reduced hardness and adhesiveness is expected to be easier to mix, transport, and to have less slimy mouth feel etc., which makes it easier for use in the manufacture of a wide variety of products. In addition, fiber contents (both soluble and insoluble fiber) can be measured according to known methods in the art. Reduced water uptake rate makes the modified psyllium easier to disperse and mix with other ingredients.

Water absorbing capacity was determined gravimetrically according to the previous method described by Elizalde et al (1996), J. of Food Science 61:407-409, with some modification. Briefly, all samples were equilibrated in a 60° C. oven for 48 hours. Then samples were transferred into a 97% relative humidity (RH) chamber and exposed to moisture for 10 min. The dry matter and the absolute amount of absorbed water were determined. All measurements were made in triplicate. The results were expressed as “mean±SD” in mg water absorbed by per gram of psyllium per minutes (mg/g/min).

Gelling properties were analyzed using a TA-XT2 texture analyzer (Texture Technologies Corp, Scarsdale, N.Y.), with a 35 mm diameter probe [Paraskevopoulou and Kiosseoglou, (1997) Journal of Food Science 62:208-211]. 1.50 g of psyllium was added into 30 ml distilled deionized water and stirred for 30 seconds at highest speed. After setting for 24 hours, gel samples were subjected to a double compression test. Measurements were performed with a pretest speed of 2.0 mm/sec, a test speed of 5.0 mm/sec, a post test speed of 5.0 mm/sec, and a distance of 6 mm. All measurements were made in triplicate. The results were expressed as “mean±SD” in gram force for hardness and adhesiveness as shown in Tables 1-11. The results are also shown in FIG. 16 for comparison with the control and raw psyllium.

### EXAMPLES

The following examples are provided for illustrative purposes, and are not intended to limit the scope of the invention as claimed herein. Any variations in the exemplified articles which occur to the skilled artisan are intended to fall within the scope of the present invention.

### Example 1

960 units of Viscozyme L (Novo North America, Inc.) were added to raw psyllium. This enzyme preparation contains 100 PBG/g with a density of 1.2. This enzyme contains xylanase, arabinase, cellulase, Beta-glucanase, and hemicellulase activities. The reaction was carried out at ambient temperature (20° C.) for 140 hr, and the enzyme was inactivated by heating in a microwave oven for 1.5 min. The results are shown in Table 2. Soluable and insolable fiber contents in modified and raw psyllium were measured using a commercial kit purchased from Megazyme International Ireland Ltd. (Wicklow, Ireland) according to the procedure of Lee et al. (1995) J. of AOAC International, 78(3):724-729.

### Example 2

The reaction conditions of Example were followed, except that 1800 units of Viscozyme L were added. The results are shown in Table 3.

### Example 3

1,100 units of Shearzyme L (8 ml of Shearzyme L, obtained from Novo North America, Inc. and 7 ml of 50 mM acetate buffer at pH 4.8) were added and mixed into the raw psyllium. This enzyme preparation contains 500 Fungal Xylanase Units per gram (FXU/g) with a density of 1.1-1.2g/ml. The enzyme is a purified xylanase, virtually free of amylase and protease activities. The reaction was carried out at ambient temperature (20° C.), and the reaction was terminated by treating the mixture with 100 ml of ethanol at ambient temperature for 30 minutes. The ethanol was removed by drying overnight at ambient temperature or vacuum filtration. The solid was washed with 30 ml acetone and air-dried overnight at ambient temperature. Analytical results are shown in Table 4.

### Example 4

5,500 units of Shearzyme L (15 ml of commercial Shearzyme L) were used in the reaction according to the procedure described in Example 3. The results are shown in Table 5.

### Example 5

20,000 units of Pentopan Mono BG (Novo North America, Inc) were mixed into the raw psyllium. This enzyme contains 2,500 FXU/g. The enzyme is a purified endo-1,4-xylanase (pentosanase). The reaction temperature was 20° C. (ambient temperature), and the enzyme was inactivated by heating in a microwave oven for 1.5 min. Analytical results are shown in Table 6.

### Example 6

5,000 units of Pentopan Mono BG (Novo North America, Inc.) and 3,000 units of Shearzyme L (2 g of Pentopan Mono BG, 5 ml of Shearzyme L, and 10 ml of 50 mM, pH 4.8 acetate buffer) were mixed into the raw psyllium. The reaction conditions were as described in Example 5. Analytical results are shown in Table 7.

### Example 7

5,000 units of Pentopan Mono BG and 3,000 units of Shearzyme L (2 g of Pentopan Mono BG, 5 ml of Shearzyme L and 10 ml of double distilled water were mixed into the raw psyllium. The reaction conditions were as described in Example 5. Analytical results are shown in Table 8.

### Example 8

5,000 units of Pentopan Mono BG and 3,000 units of Shearzyme L (2 g of Pentopan Mono BG, 5 ml of Shearzyme L and 10 ml of 50 mM acetate, pH 4.8) were mixed into the raw psyllium. The reaction was carried out as described in Example 5. Analytical results are shown in Table 9.

### Example 9

240 units of Viscozyme L and 1,800 units of Shearzyme L were used (2 ml of Viscozyme L, 3 ml of Shearzyme L, and 10 ml of 50 mM acetate, pH 4.8). The results are shown in Table 10.

### Example 10

5,000 units of Pentopan Mono BG and 3,000 units of Shearzyme L (2 g of Pentopan Mono BG, 5 ml of Shearzyme L and 10 ml of 50 mM acetate, pH 4.8) were mixed into the raw psyllium. Reaction was conducted at 50° C., and the reaction was terminated by heating in a microwave oven for 1.5 min. Analytical results are shown in Table 11.

TABLE 1
(Controls)

| | | In- | Rate of H₂O | | Adhesive- |
|---|---|---|---|---|---|
| Sample | Soluble | soluble | absorbing | Hardness | ness |
| ID | fiber % | fiber % | mg/g Psy/min. | g | g |
| Raw- | 77.17 | 12.06 | 1.18 ± 0.05 | 218.5 ± 23.9 | 53.5 ± 2.7 |
| Psyllium | | | | | |
| Control I | 72.87 | 12.38 | 0.95 ± 0.01 | 266.1 ± 17.3 | 53.8 ± 4.3 |
| Control II | 78.46 | 12.98 | 1.32 ± 0.01 | 216.8 ± 16.4 | 53.7 ± 1.1 |
| Control III | 81.06 | 11.60 | 1.04 ± 0.06 | 177.5 ± 9.9 | 47.5 ± 2.6 |

TABLE 2

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 73.13 | 11.91 | 0.63 ± 0.03 | 192.9 ± 1.4 | 43.9 ± 1.0 | 50.4 |

TABLE 3

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 68.33 | 12.27 | 0.53 ± 0.04 | 133.4 ± 6.6 | 38.2 ± 0.7 | 53.6 |

TABLE 4

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 79.18 | 12.67 | 1.02 ± 0.10 | 137.7 ± 26.0 | 29.0 ± 6.7 | 50.4 |

TABLE 5

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 73.09 | 12.51 | 0.61 ± 0.03 | 93.9 ± 10.1 | 20.7 ± 3.3 | 45.8 |

TABLE 6

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 67.62 | 11.74 | 0.91 ± 0.08 | 150.3 ± 5.1 | 43.9 ± 1.3 | 56.2 |

TABLE 7

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 71.38 | 13.54 | 0.60 ± 0.02 | 83.7 ± 1.1 | 18.6 ± 0.1 | 51.9 |

TABLE 8

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 72.79 | 12.72 | 0.72 ± 0.02 | 88.2 ± 1.8 | 18.7 ± 0.3 | 52.7 |

TABLE 9

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 71.14 | 13.02 | 0.93 ± 0.05 | 77.9 ± 3.6 | 16.7 ± 1.2 | 55.2 |

TABLE 10

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 74.06 | 13.22 | 0.71 ± 0.06 | 188.6 ± 6.4 | 40.0 ± 1.6 | 48.9 |

TABLE 11

| Soluble | Insoluble | Rate of H₂O | | Adhesive- | |
|---|---|---|---|---|---|
| fiber | fiber | absorbing | Hardness | ness | Yield |
| % | % | mg/g Psy/min. | g | g | g |
| 64.65 | 12.91 | 0.54 ± 0.03 | 84.5 ± 2.3 | 18.1 ± 0.8 | 49.0 |

The results shown in Tables 1-11 demonstrate that the modified psyllium prepared according to the solid-state enzyme reaction indeed exhibits improved functionality. In all examples, the modified psyllium showed a substantially lower rate of water absorption, decreased hardness, and adhesiveness, compared to the control and raw psyllium.

Since the extent of gel hardness and adhesiveness of a psyllium preparation is used to predict certain properties such as slimy mouth feel, gelling ability, ease of miscibility/dispensability etc. of a given psyllium, the modified psyllium of the invention with reduced hardness, adhesiveness and rate of water uptake should improve processing qualities (i.e., easy to disperse, easy to mix with other ingredients) in manufacturing various psyllium-containing products. A product containing the modified psyllium of the invention should have less slimy mouth feel.

Because the solid-state reaction is not carried out in the presence of acid or salt or solvent, the modified psyllium of the present invention should be free of residual salt, acid or solvent and any other additives which may be present in a psyllium preparation prepared by other prior art methods.

All references cited in the present application are incorporated in their entirety herein by reference to the extent not inconsistent herewith.

We claim:

1. A method of producing modified psyllium, comprising: treating raw psyllium with an effective amount of an enzyme capable of modifying said raw psyllium in a solid-state reaction such that the resultant modified psyllium exhibits less gelling properties and water uptake than said raw psyllium.

2. The method of claim 1 wherein the enzyme is selected from the group consisting of xylanases, cellulases, hemicellulases, pentosanases, beta-glucanases, and mixtures of thereof.

3. The method of claim 1 wherein the enzyme is a xylanase.

4. The method of claim 1 wherein said solid-state reaction is carried out at 20-55° C.

5. The method of claim 1, further comprising the step of inactivating the enzyme.

6. The method of claim 1 wherein said raw psyllium is 90% pure, 100 mesh psyllium.

7. The modified psyllium prepared by the method of any one of claims 1 to 6.

8. A pharmaceutical composition comprising the modified psyllium prepared by the method of any one of claims 1 to 6.

9. A food product comprising the modified psyllium prepared by the method of any one of claims 1 to 6.

10. A beverage comprising the modified psyllium prepared by the method of any one of claims 1 to 6.

11. A drink mix comprising the modified psyllium prepared by the method of any one of claims 1 to 6.

12. A livestock feed comprising the modified psyllium prepared by the method of any one of claims 1 to 6.

13. A bulk laxative comprising the modified psyllium prepared by the method of any one of claims 1 to 6.

14. A method of preparing an edible psyllium-containing product comprising: blending raw psyllium with at least one edible ingredient to form a blend; and adding to said blend an effective amount of an enzyme capable of modifying raw psyllium in a solid-state reaction such that the resultant modified psyllium exhibits less hardness and/or adhesiveness than said raw psyllium.

15. The method of claim 14, further comprising the step of inactivating the enzyme.

16. The method of claim 14, wherein the enzyme is selected from the group consisting of xylanases, cellulases, hemicellulases, pentosanases, beta-glucanases, and mixtures of thereof.

17. The method of claim 14, wherein the enzyme is a xylanase.

18. The method of claim 14, wherein said solid-state reaction is carried out at 20-55° C.

19. A method of lowering serum cholesterol in mammals, comprising orally administering a sufficient amount of the modified psyllium prepared by the method of any one of claims 1 to 6 to a mammal.

20. A method of lowering serum triglycerides in mammals, comprising orally administering a sufficient amount of the modified psyllium prepared by the method of any one of claims 1 to 6 to a mammal.

21. A method of lowering serum LDL in mammals, comprising orally administering a sufficient amount of the modified psyllium prepared by the method of any one of claims 1 to 6 to a mammal.

22. A method of producing a bulk laxative for use in mammals, comprising orally administering a sufficient amount of the modified psyllium prepared by the method of any one of claims 1 to 6 to a mammal.
